(19) United States
(12) Patent Application Publication
(10) Pub. No.: US 2023/0330153 A1
(43) Pub. Date: Oct. 19, 2023

(54) ENGINEERED SKIN CONSTRUCT OVERLAY OF AUTOGRAFT

(71) Applicant: Stratatech Corporation, Madison, WI (US)

(72) Inventors: ALLEN R. COMER, Madison, WI (US); JANICE SMIELL, Madison, WI (US); B. LYNN ALLEN-HOFFMANN, Madison, WI (US); MARY LOKUTA, Madison, WI (US); RANDI RUTAN, Madison, WI (US)

(21) Appl. No.: 18/014,951

(22) PCT Filed: Jul. 8, 2021
(86) PCT No.: PCT/US2021/040892

§ 371 Date: Jan. 6, 2023

Related U.S. Application Data
(60) Provisional application No. 63/049,447, filed on Jul. 8, 2020.

(51) Int. Cl. A61L 27/60; A61K 38/39; A61P 17/02; A61L 27/36; A61L 27/38; A61L 27/24; A61L 27/56; A61K 35/36

(52) U.S. Cl. CPC A61K 35/36; A61K 38/39; A61L 27/24; A61L 27/362; A61L 27/3813; A61L 27/56; A61L 27/60; A61P 17/02; A61L 2430/34

(57) ABSTRACT

The present disclosure provides methods for treating wounds by applying a meshed autograft and skin substitute overlay wherein the skin substitute is an organotypic human skin equivalent comprising NIKS® cells.

### CROSS-REFERENCE TO RELATED APPLICATIONS

This application claims priority to U.S. Provisional Pat. Application No. 63/049,447, filed Jul. 8, 2020, herein incorporated by reference in its entirety.

### FIELD

The present disclosure encompasses methods for treating wounds by applying a meshed autograft and skin substitute overlay.

### BACKGROUND

The current standard of care for closure of many deep partial-thickness (DPT) or full-thickness (FT) wounds is autografting, i.e., the surgical harvest of healthy skin from a donor site and transplantation of the harvested donor tissue to a recipient site in the same individual. While this method is effective in achieving closure of the original wound, availability of sites for the harvest of donor skin may be limited, and closure of large areas of injury may necessitate sequential re-harvest of available donor sites. Reduction in the amount of donor tissue necessary to achieve complete wound closure is of clinical benefit, as the harvest of a donor site creates an additional partial-thickness wound which adds to the patient’s overall wound burden and is itself painful and susceptible to infection, scarring and chronic impairment of skin function, and can convert to a FT wound.

Autograft meshing, i.e., the expansion of harvested tissue through a meshing device to increase its surface area, is one strategy used to reduce the amount of donor tissue. However, meshed grafts heal with more scarring due to the secondary healing of the interstices and are therefore less aesthetically appealing compared with unmeshed skin grafts.

As a result, there is an urgent need for alternatives that reduce the area of donor site needed to achieve closure of DPT and FT wounds.

### SUMMARY

In an aspect, the present disclosure encompasses a method for treating a full-thickness wound in a subject. The method comprises applying to the wound an autograft with a mesh ratio of about 2:1 to about 8:1 (“treatment autograft”) and a skin substitute overlay, wherein the skin substitute is an organotypic human skin equivalent comprising NIKS cells.

In another aspect, the present disclosure encompasses a method for obtaining closure of a full-thickness wound in a subject. The method comprises applying to the wound an autograft with a mesh ratio of about 2:1 to about 8:1 (“treatment autograft”) and a skin substitute overlay, wherein the skin substitute is an organotypic human skin equivalent comprising NIKS cell and wherein wound closure is about 90% re-epithelialization or greater.

In yet another aspect, the present disclosure encompasses a method for improving an outcome of wound healing in a subject. The method comprises: applying to a full-thickness wound of a subject an autograft with a mesh ratio of about 2:1 to about 8:1 (“treatment autograft”) and a skin substitute overlay, wherein the skin substitute is an organotypic human skin equivalent comprising NIKS cells, wherein the improved outcome is a reduction in the incidence of autografting, decreased donor skin utilization, decreased pain, an improvement in one or more cosmetic quality of the closed wound, decreased treatment associated adverse events, decreased infection rate, decreased wound-infection related events, decreased donor-site morbidities, reduced health care resource utilization, improved quality of life, or combinations thereof, as measured by an observer or by the subject in comparison to a comparable wound treated with autograft alone (“comparator autograft”), wherein the comparator autograft has a mesh ratio that is the same or less than the treatment autograft.

Other aspects and iterations of the invention are described more thoroughly below.

### DETAILED DESCRIPTION

In one aspect, the present disclosure provides methods for treating a variety of wounds, the method comprising applying to the wound a meshed autograft and a skin substitute overlay. The term “skin substitute overlay” indicates that a skin substitute is positioned over something. In the present disclosure, a skin substitute is always applied over an autograft, meaning the autograft is positioned between the wound and the skin substitute. Treatment facilitates wound closure and may also reduce the total area of autograft needed to achieve wound closure, reduce the incidence of autograft for the subject (e.g., eliminate or decrease subsequent autografting), and improve other outcomes for the subject.

Accordingly, in another aspect, the present disclosure provides methods for wound closure, the method comprising applying to a wound of a subject a meshed autograft and a skin substitute overlay. As used herein, the term “wound closure” means ≥90% re-epithelialization of the wound surface with absence of drainage. In another aspect, the present disclosure provides methods for improving an outcome of wound healing, the method comprising applying to a wound of a subject a meshed autograft and a skin substitute overlay. Non-limiting examples of improved outcomes for a subject include decreased pain, a reduction in the incidence of autografting, decreased donor skin utilization, an improvement in one or more cosmetic quality of a closed wound, decreased treatment associated adverse events, decreased infection rate, decreased wound-infection related events, decreased donor-site morbidities, reduced health care resource utilization, or combinations thereof. Cosmetic qualities of a closed wound that may improve include but are not limited to pigmentation or coloring, scar thickness, pliability, stiffness, itching, vascularity, and overall appearance. Donor-site morbidities include but are not limited to tenderness, pain, cold-sensitivity, scarring in general or more particularly hypertrophic scarring, infection, conversion from a split-thickness wound to a complex wound or a full thickness wound, etc. Decreased donor site utilization refers to a decrease in the total surface of donor site(s) harvested from a subject. A reduction in the incidence of autografting may be an elimination of, or a decrease in, autografting after the initial application. In some embodiments, an improvement in an outcome is in comparison to a comparable wound treated only with a meshed autograft, wherein both autografts have the same mesh ratio or the comparator autograft has a smaller mesh ratio. As used herein, the term “comparable wound” has a substantially similar area, wound bed composition (e.g., fat, fascia, or muscle), and kinesiologic stressors as a treatment wound.

Methods of the present disclosure comprise applying a meshed autograft to a wound and then applying a skin substitute overlay, wherein the skin substitute is an organotypic human skin equivalent comprising NIKS cells. The size of the skin substitute is tailored to substantially cover the meshed autograft. Typically, the meshed autograft has a mesh ratio of at least 2:1. The autograft and skin substitute may be applied sequentially. For example, first the autograft is applied to the wound and then the skin substitute is applied over the autograft. A suitable alternative to the sequential application of a meshed autograft and a skin substitute is the application of a pre-formed “sandwich” comprised of the meshed autograft and the skin substitute. The autograft and skin substitute overlay may be applied to the wound within hours or days of the wound’s formation, within the first or second week of the wound’s formation, or even later. Wounds are typically prepared for treatment by methods known in the art, for instance cleansing with soap and water, surgical excision (fascial excision or tangential excision) if clinically indicated and feasible, etc. The location of the wound is not limiting. For instance, a wound may be on an arm, a leg, a torso, a face, a head, a neck, a hand, a foot, a buttock, over joints, etc. In some embodiments, the method consists of a single application of the skin substitute overlay. In other embodiments, the method comprises 2 or more applications of the skin substitute overlay.

Methods of the present disclosure are useful for a variety of wounds, in particular deep partial-thickness wounds and full-thickness wounds.

Other aspects of the disclosure are described in further detail below.

### 1. Autograft

Methods of the present disclosure require application of a meshed autograft to a wound. Autografting is the surgical harvest of healthy skin from a donor site and transplantation of the harvested donor tissue to a recipient site in the same individual. The term “autograft” refers to the harvested donor tissue. An autograft may be a split-thickness autograft or a full-thickness autograft. In preferred embodiments, an autograft is a full-thickness autograft.

A “meshed tissue” refers to a tissue that is perforated with slits, typically by a meshing device. The size and number of slits affect drainage and potential expansion. A mesh ratio, abbreviated as “m”, is used in the art to indirectly describe the amount of meshing, and is defined as the expected size of an expanded tissue as compared to its size before expansion. A mesh ratio of 1:1 indicates a tissue (e.g., an autograft) is meshed but no expansion is expected. Tissues with a higher mesh ratio will have a greater number and/or size of slits. A meshed tissue can then be expanded by a user prior to application to about the expected size in order to increase the surface area covered by the meshed tissue.

A meshed autograft of the present disclosure has a mesh ratio of at least 1:1, typically at least 2:1. In some embodiments, a method of the present disclosure comprises applying to a wound an autograft with a mesh ratio of at least 2:1. For instance, the autograft may have a mesh ratio of 2:1, or about 3:1, about 4:1, about 5:1, about 6:1, about 7:1, about 8:1, or more. In some embodiments, a method of the present disclosure comprises applying to a wound an autograft with a mesh ratio of about 2:1 to about 8:1 and then applying a skin substitute overlay. In some embodiments, a method of the present disclosure comprises applying to a wound an autograft with a mesh ratio of about 3:1 to about 8:1and then applying a skin substitute overlay. In some embodiments, a method of the present disclosure comprises applying to a wound an autograft with a mesh ratio of about 4:1 to about 8:1 and then applying a skin substitute overlay. In some embodiments, a method of the present disclosure comprises applying to a wound an autograft with a mesh ratio of about 2:1 to about 6:1 and then applying a skin substitute overlay. In some embodiments, a method of the present disclosure comprises applying to a wound an autograft with a mesh ratio of about 3:1 to about 6:1 and then applying a skin substitute overlay. In some embodiments, a method of the present disclosure comprises applying to a wound an autograft with a mesh ratio of about 4:1 to about 6:1and then applying a skin substitute overlay.

Methods for harvesting split-thickness and full-thickness autografts, autograft meshing, autograft placement and fixation are well-known in the art.

### II. Skin Substitute

Methods of the present disclosure also require application of a skin substitute overlay, wherein the skin substitute is an organotypic human skin equivalent comprising NIKS cells. Accordingly, the skin substitute is an engineered (non-natural), bilayer tissue designed to mimic natural human skin with both an inner dermis-like layer and an outer epidermis-like layer. For example, production of the skin substitute by organotypic culture produces a well-developed epidermal layer of fully-stratified human keratinocytes that exhibits barrier function comparable to that of intact human skin.

The inner dermis-like layer is also referred to as a “dermal equivalent.” Suitable dermal equivalents are a matrix comprising gelled collagen and human dermal fibroblasts. In exemplary embodiments, the human dermal fibroblasts are primary normal human dermal fibroblasts. The collagen present in the dermal equivalent may include type I murine collagen. Alternatively, the only collagen present in the dermal equivalent may be produced by cells of the skin substitute (e.g., human dermal fibroblasts). The matrix may further comprise additional biomolecules produced by the cells contained therein. In an exemplary embodiment, the dermal layer is composed of normal human dermal fibroblasts embedded within an extracellular matrix produced and organized by the fibroblasts. For the avoidance of doubt, in this embodiment, there is no non-human collagen in the dermal layer. In another exemplary embodiment, the dermal layer is composed of normal human dermal fibroblasts embedded in a gelled-collagen matrix that contains purified murine type I collagen. For the avoidance of doubt, in this embodiment, although the rat-tail tendon type I collagen is gelled to give the dermal layer its primary structure, the normal human dermal fibroblasts embedded therein may produce and contribute collagen (and other biomolecules) to the matrix.

The outer epidermis-like layer comprises NIKS cells. NIKS® cells were deposited with the ATCC (CRL-12191) and are described in further detail in U.S. Patent No. 5,989,837 and U.S. Pat. No. 6,964,869, the disclosures of which are incorporated herein by reference. The phrase “an organotypic human skin equivalent comprising NIKS cells” encompasses NIKS® cells engineered to express a variety of exogenous nucleic acids that may provide a beneficial effect in wound closing (e.g., encode a protein that directly or indirectly promotes wound healing). Expressly contemplated are NIKS® cells engineered to express an exogenous gene encoding a VEGF protein (e.g., VEGF-A, etc.), an exogenous gene encoding a hypoxia-inducible factor (e.g., HIF-1A, etc.), an exogenous gene encoding an angiopoietin (e.g., ANGPT1, etc.), an exogenous gene encoding a cathelicidin peptide or a cleavage product thereof (e.g., hCAP-18, etc.), an exogenous gene encoding a beta-defensin (e.g., hBD-3, etc.), an exogenous gene encoding a keratinocyte growth factor (e.g., KGF-2, etc.), an exogenous gene encoding a tissue inhibitor of metalloproteinases (e.g., TIMP-1, etc.), an exogenous IL-12 gene, as well exogenous nucleic acid sequences encoding other antimicrobials, growth factors, transcription factors, interleukins and extracellular matrix proteins. As non-limiting examples, see for instance, U.S. Patent Nos. 7498167, 7915042, 7807148, 7988959, 8808685, 7674291, 8092531, 8790636, 9526748, 9216202, and 9163076, and US 20190030130, the disclosures of which are incorporated herein by reference. Skin substitutes comprising NIKS cells engineered to express an exogenous nucleic acid encoding a desired protein produce a greater amount of that protein (e.g., at least 10%, at least 20%, at least 30%, etc. more) than a skin substitute comprising NIKS cells that do not contain the exogenous nucleic acid.

The viable cells of the skin substitute (e.g., fibroblasts, NIKS cells, etc.) are metabolically active and secrete a spectrum of growth factors, chemotactic factors, cytokines, inflammatory mediators, enzymes, and host defense peptides that condition the wound bed, promote tissue regeneration and repair, and reduce infection. In one embodiment, the skin substitute is an organotypic human skin equivalent comprising NIKS cells, wherein the cells of the skin substitute secrete, relative to an autograft, at least about 10% to about 15%, about 15% to about 25%, about 25% to about 50%, about 50% to about 75%, about 75% to about 85%, about 85 to about 95%, about 90% to 100%, about 95% to about 105%, about 100% to 110%, about 105% to about 115%, about 110% to 120%, about 115% to about 125%, about 120% to 130%, about 125% to about 135%, or more, of growth factors, chemotactic factors, cytokines, inflammatory mediators, enzymes, host defense peptides, and combinations thereof that condition the wound bed, promote tissue regeneration and repair, and/or reduce infection. In another embodiment, wherein the cells of the skin substitute secrete, relative to an autograft, at least about 10% to about 15%, about 15% to about 25%, about 25% to about 50%, about 50% to about 75%, about 75% to about 85%, about 85 to about 95%, about 90% to 100%, about 95% to about 105%, about 100% to 110%, about 105% to about 115%, about 110% to 120%, about 115% to about 125%, about 120% to 130%, about 125% to about 135%, or more, of growth factors, chemotactic factors, cytokines, inflammatory mediators, enzymes, host defense peptides, and combinations thereof, further wherein use of the skin substitute results in an improved outcome of decreased infection (e.g., a 1%, 5%, 10%, 15%, 20%, 25%, 30%, 35%, 40%, 50%, 55%, 60%, 70%, 75%, 80%, 85%, 90%, 95%, or 100% decrease in infection), as compared to an autograft. In certain examples of the above embodiments, the growth factors, chemotactic factors, cytokines, inflammatory mediators, enzymes, and host defense peptides may be selected from the group consisting of a VEGF protein (e.g., VEGF-A, etc.), a hypoxia-inducible factor (e.g., HIF-1A, etc.), an angiopoietin (e.g., ANGPT1, etc.), a cathelicidin peptide or cleavage product thereof (e.g., hCAP-18, etc.), a beta-defensin (e.g., hBD-3, etc.), a keratinocyte growth factor (e.g., KGF-2, etc.), a tissue inhibitor of metalloproteinases (e.g., TIMP-1, etc.), and combinations thereof.

Suitable manufacturing processes for producing a skin substitute that is an organotypic human skin equivalent comprising NIKS® cells have been previously described in the art. See, for instance, U.S. Pat. Nos. 7498167, 7915042, 7807148, 7988959, 8808685, 7674291, 8092531, 8790636, 9526748, 9216202, 9163076 and 10091983, and US 20190030130, the disclosure of which are incorporated by reference in their entirety. Advantageously, skin substitutes produced as described above have excellent handling characteristics that enable them to be meshed, sutured, stapled or secured with an adhesive, in the same manner as autologous skin grafts, cadaver allografts and xenografts.

The skin substitute may be optionally meshed. In some embodiments, the skin substitute has a mesh ratio of about 1:1 or more (e.g., about 1.5:1, about 2:1, about 2.5:1, about 3:1, etc.). In some embodiments, the skin substitute is not meshed.

In an exemplary embodiment, the skin substitute is StrataGraft®. In another exemplary embodiment, the skin substitute is an ExpressGraft™ tissue.

### III. Wound

The terms “skin wound” and “wound” are used interchangeably herein, and refer to a breach in continuity of skin. Skin wounds are defined in the art as acute or chronic. An acute wound has normal wound physiology, and healing is anticipated to progress through the normal stages of wound healing. A chronic wound is defined as one that has failed to pass through the normal healing process – i.e., does not heal in an orderly set of stages and in a predictable amount of time (e.g., a three to four week period of time). In some embodiments, a meshed autograft and a skin substitute overlay are applied to a chronic wound. In some embodiments, a meshed autograft and a skin substitute overlay are applied to an acute wound.

Acute wounds are generally classified based on the mode of infliction and/or causative agent. Methods of the present disclosure may be used to treat a variety of acute wound types including, but not limited to, surgical wounds, burn wounds, bite wounds, puncture wounds, sharp cuts, lacerations, etc. In exemplary embodiments, a meshed autograft and a skin substitute overlay are applied to a surgical wound. Non-limiting examples of surgical wounds that may be treated include donor sites for grafts, post-laser surgery wounds, post-podiatric procedure wounds, cosmetic surgery wounds, cancer excisions, wounds generated by the treatment of scar contractures, etc. In other exemplary embodiments, a meshed autograft and a skin substitute overlay are applied to a burn wound. In some examples, the wound is from a thermal burn. A “thermal burn” refers to a burn cause by fire, hot objects, steam, or hot liquids. In some examples, the wound is from an electrical burn. An “electrical burn” refers to a burn caused by contact with electrical sources, including a lightning strike. In some examples, the wound is from a radiation burn. A “radiation burn” refers to a burn caused by prolonged exposure to sources of UV radiation such as sunlight (e.g., sunburn), tanning booths, or sunlamps or by X-rays, radiation therapy or radioactive fallout. In some examples, the wound is from a chemical burn. A “chemical burn” refers to a burn caused by contact with highly acidic or basic substances. In some examples, the wound is from a friction burn. A “friction burn” refers to a burn caused by friction between the skin and hard surfaces, such as roads, carpets, floors, etc.

Chronic wounds have physiologically impaired wound healing due to disruption of the wound healing cycle as a result of impaired angiogenesis, innervation, or cellular migration, among other reasons known in the art. Methods of the present disclosure may be used to treat a variety of chronic wounds including, but not limited to, nonhealing or infected surgical or traumatic wounds, venous ulcers, pressure ulcers, diabetic foot ulcers, and ischemic ulcers.

In each of the above embodiments, the acute wound or the chronic wound may be a full-thickness wound, a partial-thickness wound, or a complex wound that contains intact dermal elements. Methods of the present disclosure are particularly useful for full-thickness wounds, as well as deep partial thickness wounds. The term “full-thickness” in the context of a skin wound is defined as penetrating the epidermis and dermis but not extending beyond the subcutaneous tissue. The term “partial-thickness” in the context of a skin wound is defined as penetrating the epidermis and extending into, but not penetrating, the dermis. The dermis itself is divided into two regions, the uppermost being the papillary region. This area is composed mostly of connective tissue and serves only to strengthen the connection between the epidermis and the dermis. Partial-thickness wounds that only extend down to this layer of the skin are considered superficial partial thickness wounds. The reticular region of the dermis contains not only connective tissue, but hair follicles, sebaceous and sweat glands, cutaneous sensory receptors, and blood vessels. Damage to this layer of the skin is classified as a deep partial-thickness wound, and can lead to significant scarring. The term “complex wound” in the context of a skin wound means the wound has different depths or etiologies across it. The term “containing intact dermal elements” means there are portions of skin structures (e.g., hair follicles, sweat glands, etc.) remaining in the bed of the wound that can provide a source of keratinocytes or keratinocyte precursor cells for migration to the surface of the wound bed. The terms “partial-thickness” and “full-thickness” are mutually exclusive but may coincide across a complex wound. For instance, a “complex wound that contains intact dermal elements” may refer to a wound that has both full-thickness characteristics (e.g., no intact dermal elements, etc.) and partial-thickness characteristics (e.g., intact dermal elements, etc.).

In the management of burn wounds, it is known that an acute burn wound may continue to progress over the first few days leading to a change in classification of the wound. As a non-limiting example, a superficial burn wound may progress to a partial-thickness burn wound, a complex burn wound that contains intact dermal elements, or a full-thickness burn wound over a period of days. Alternatively, or in addition to change in the depth of the burn, an increase in burn surface area may occur. This phenomenon is referred to as “burn wound conversion,” and the wound is then referred to as “a secondary burn wound.” This progression is not considered a failure to pass through the normal healing process. As such, in some embodiments, an acute wound may be a secondary burn wound.

Burn wounds have also been classified by the degree of the burn. A third degree burn is a full-thickness burn wound and a second degree burn is a partial thickness burn wound. However, many burn wounds are complex wounds, with different depths across the entirety of the wound. In clinical practice, these wounds are often classified based on the most severe aspect of the wound when using the degree classification system.

In the above embodiments, a wound may be less than 200 cm². For instance, the wound may be less than 150 cm², less than 100 cm², less than 50 cm², less than 10 cm², or less than 1 cm². In some embodiments, the wound may be about 1 cm² to 199 cm². For instance, the wound may be 0.5 cm² to about 195 cm², 0.5 cm² to about 150 cm², 0.5 cm² to about 100 cm², or 0.5 cm² to about 50 cm². Alternatively, the wound may be about 5 cm² to about 195 cm², about 5 cm² to about 150 cm², about 5 cm² to about 100 cm², or about 5 cm² to about 50 cm². Alternatively, the wound may be about 50 cm² to about 195 cm², about 50 cm² to about 150 cm², or about 50 cm² to about 100 cm².

Alternatively, in the above embodiments, a wound may be greater than or equal to 200 cm². For instance, the wound may be greater than or equal to 250 cm², greater than or equal to 300 cm², greater than or equal to 350 cm², or greater than or equal to 400 cm². The wound may also be greater than or equal to 500 cm², greater than or equal to 600 cm², greater than or equal to 700 cm², greater than or equal to 800 cm², greater than or equal to 900 cm², or greater than or equal to 1000 cm². The wound may also be greater than or equal to 2000 cm², greater than or equal to 3000 cm², greater than or equal to 4000 cm², greater than or equal to 5000 cm², or greater than or equal to 10,000 cm². In some embodiments, the wound may be 200 cm² to 1000 cm². For instance, the wound may be about 200 cm² to about 950 cm², about 200 cm² to about 900 cm², about 200 cm² to about 800 cm², about 200 cm² to about 700 cm², or about 200 cm² to about 600 cm². Alternatively, the wound may be about 200 cm² to about 500 cm², about 200 cm² to about 450 cm², or about 200 cm² to about 400 cm². In some embodiments, the wound may be 200 cm² to 2000 cm². For instance, the wound may be about 200 cm² to about 1950 cm², about 200 cm² to about 1900 cm², about 200 cm² to about 1800 cm², about 200 cm² to about 1700 cm², or about 200 cm² to about 1600 cm². Alternatively, the wound may be about 200 cm² to about 1500 cm², about 200 cm² to about 1450 cm², or about 200 cm² to about 1400 cm². Alternatively, the wound may be about 1000 cm² to about 1950 cm², about 1000 cm² to about 1900 cm², about 1000 cm² to about 1800 cm², about 1000 cm² to about 1700 cm², or about 1000 cm² to about 1600 cm². Alternatively, the wound may be about 1000 cm² to about 1500 cm², about 1000 cm² to about 1450 cm², or about 1000 cm² to about 1400 cm². Alternatively, the wound may be about 1000 cm² to about 15,000 cm², about 3500 cm² to about 15,000 cm², or about 5000 cm² to about 15,000 cm².

Methods of the present disclosure may be used to treat uninfected wounds or wounds that are deemed clinically infected. In embodiments where the wound is deemed clinically infected, it may be advantageous to use an organotypic human skin equivalent comprising NIKS® cells engineered to express one or more antimicrobial peptide.

Methods of the present disclosure may be particularly preferred for wounds where surgical intervention (e.g., excision and autografting) is clinically indicated, and where acute wound is a full-thickness wound or a deep partial-thickness wound.

### IV. Subject

A “subject,” as used herein, refers to a human with an acute wound or a chronic wound, as the terms are described in Section III. In some embodiments, the subject has an acute wound or a chronic wound where surgical intervention is clinically indicated (e.g., debridement and autografting). In further embodiments, the wound is a full-thickness wound or a deep partial-thickness wound. In still further embodiments, the acute would is from a burn or is a surgical wound. In still further embodiments, the acute would is from a thermal burn.

In some embodiments, a subject is 18 to 75 years in age. In other embodiments, a subject is less than 18 years in age. In still other embodiments, a subject is 65 years or older, 75 years or older, over 75 years of age.

In each of the above embodiments, the subject’s total body surface area covered by the wound or more likely by a plurality of wounds, referred to herein as “% TBSA,” may vary. In some embodiments, the % TBSA is at least 1%, at least 2%, or at least 5%. In some embodiments, the % TBSA is greater than 5%, greater than 10%, greater than 15%, or greater than 20%. In some embodiments, the % TBSA is greater than 25%. In some embodiments, the % TBSA is about 1% to about 90%, or about 1% to about 85%. In some embodiments, the % TBSA is about 1% to about 60%. In some embodiments, the % TBSA is about 1% to about 50%. In some embodiments, the % TBSA is about 5% to about 50%. In some embodiments, the % TBSA is about 10% to about 50%. In some embodiments, the % TBSA is about 15% to about 50%. In some embodiments, the % TBSA is about 20% to about 50%. In some embodiments, the % TBSA is about 25% to about 50%. In some embodiments, the % TBSA is about 10% to about 90%. In some embodiments, the % TBSA is about 15% to about 90%. In some embodiments, the % TBSA is about 20% to about 90%. In some embodiments, the % TBSA is about 25% to about 90%. In some embodiments, the % TBSA is about 10% to about 85%. In some embodiments, the % TBSA is about 15% to about 85%. In some embodiments, the % TBSA is about 20% to about 85%. In some embodiments, the % TBSA is about 25% to about 85%.

A “full-thickness % TBSA” indicates a subject’s total body surface area covered by full-thickness wound(s). In various embodiments, a subject has a full-thickness % TBSA of at least 1%, or at least 5%. For instance, a subject may have a full-thickness % TBSA of 1%, 2%, 3%, 4%, 5%, 10%, 15%, 20%, 25%, 30%, 35%, 40%, 45%, 50%, 55%, 60%, 65%, 70% or more. In some embodiments, a subject’s full-thickness % TBSA is the same, or about the same, as the % TBSA. In other embodiments, a subject’s full-thickness % TBSA is less than the % TBSA. In some examples, a subject may have a % TBSA of at least 10% and a full-thickness % TBSA of at least 2% or at least 5%. In some examples, a subject may have a % TBSA of at least 20% and a full-thickness % TBSA of at least 5%, at least 10%, or at least 15%. In some examples, a subject may have a % TBSA of at least 25% and a full-thickness % TBSA of at least 5%, at least 10%, at least 15%, or at least 20%. In some examples, a subject may have a % TBSA of at least 30% and a full-thickness % TBSA of at least 5%, at least 10%, at least 15%, or at least 20%. In some examples, a subject may have a % TBSA of at least 40% and a full-thickness % TBSA of at least 5%, at least 10%, at least 15%, at least 20%, at least 25%, or at least 30%. In some examples, a subject may have a % TBSA of at least 50% and a full-thickness % TBSA of at least 5%, at least 10%, at least 15%, at least 20%, at least 25%, at least 30%, at least 35%, or at least 40%.

Methods of the present disclosure are suitable for a variety of subjects, including vulnerable subjects. Non-limiting examples of vulnerable subjects include but are not limited to subjects with limited surface area available for donor sites, subjects for whom harvest of donor sites is contraindicated, subjects with a high Baux score, subjects that are not hemodynamically stable, subjects that are at risk for delayed or impaired wound healing, or any combination thereof. Non-limiting examples of vulnerable subjects with limited surface area available for donor sites include subjects with a high % TBSA, such as a % TBSA of about 25% or greater, about 30% or greater, or about 35% or greater; subjects 10 years of age or younger, 5 years of age or younger, or 1 year of age or younger; and subjects 65 years of age or older, 70 years of age or older, 75 years of age or older, or 80 years of age or older. A vulnerable subject with a high Baux score typically has a Baux score of 100 or more. Non-limiting examples of subjects at risk for delayed or impaired wound healing include subjects with thinning dermis, inhalation injury or other coexisting injuries, a ≥ 25% TBSA, diabetes, infection (typically at the wound site but may be elsewhere), obesity, medications known to impair wound healing, a smoking habit, excessive alcohol drinking, low blood pressure, vascular disease, edema, cancer, malnutrition, a history of hypertrophic scarring, or any combination thereof. A vulnerable subject with thininng dermis typically has a dermal thickness less than 2 mm.

### V. Wound Closure

In one aspect, the present disclosure provides a method for wound closure, the method comprising applying a meshed autograft to a wound and a skin substitute overlay, wherein the skin substitute is an organotypic human skin equivalent comprising NIKS cells. In some embodiments, the autograft has a mesh ratio of at least 2:1. In some embodiments, the autograft has a mesh ratio of about 2:1 to about 8:1, about 3:1 to about 8:1, or about 4:1 to about 8:1. In some embodiments, the autograft has a mesh ratio of about 2:1 to about 6:1, about 3:1 to about 6:1, or about 4:1 to about 6:1. In each of the above embodiments, the skin substitute may be unmeshed or have mesh ratio of about 1:1 or less.

Wounds are typically prepared for treatment by methods known in the art, for instance cleansing with soap and water, surgical excision (fascial excision or tangential excision of nonviable tissue) if clinically indicated and feasible, etc. The location of the wound is not limiting. For instance, a wound may be on an arm, a leg, a torso, a face, a head, a neck, a hand, a foot, a buttock, over joints, etc. The autograft and skin substitute overlay may be applied to the wound within hours or days of the wound’s formation, within the first or second week of the wound’s formation, or even later. When the total surface area of the wound covered by the autograft is less than the manufactured size of a skin substitute, the skin substitute may be trimmed to substantially fit the surface area and shape of the wound area covered by the autograft. When the total surface area of the wound covered by the autograft is greater than the manufactured size of a skin substitute, multiple samples may be used to substantially cover the wound area covered by the autograft by abutting the multiple samples. For the avoidance of doubt, the use of multiple samples to cover an autograft is considered a single application. In some embodiments, only a single application of the skin substitute is needed. In other embodiments, the method comprises multiple applications of the skin substitute to the wound covered by the autograft or a portion of the wound covered by the autograft on one or more different day. The timing of each subsequent application may vary. For instance a subsequent (e.g., 2^{nd}, 3^{rd,} 4^{th}, etc.) application may occur days or weeks after the preceding application. Depending upon the total surface area of the wound, allogenic tissue, autologous tissue without a skin substitute overlay, autologous cells, or a different skin substitute may be used in some embodiments in conjunction with the autograft and skin substitute overlay. For example, a wound area may be covered by abutting one or more samples of the autograft and skin substitute overlay, and one or more autograft without a skin substitute overlay.

As used herein, the term “wound closure” means ≥90% re-epithelialization of the wound surface with absence of drainage. In some embodiments, wound closure may be 90%, 91%, 92%, 93%, 94%, 95%, 96%, 97%, 98%, or 99% re-epithelialization of the wound surface with absence of drainage. In another embodiment, wound closure may be 100% re-epithelialization of the wound surface with absence of drainage, confirmed by a medical provider at two visits at least two weeks apart (“complete wound closure”). The amount of time it takes to achieve wound closure is assessed from the first application of the skin substitute (i.e., day 1). In some embodiments, wound closure may occur ≤ 3 months after the first application of the skin substitute. In some embodiments, wound closure may occur within about 1 week, about 2 weeks, about 3 weeks, about 4 weeks, about 1 month, about 2 months, or about 3 months after the first application of the skin substitute.

In still further embodiments, methods of the present disclosure for wound closure may result in durable wound closure. As used herein, the term “durable wound closure” means persistence of complete wound closure for at least 3 months after the initial observation of closure. In some examples, durable wound closure occurs ≤ 4 months after the first application of the skin substitute.

In some embodiments, the wound is an acute wound. Exemplary acute wounds are detailed in Section III, and incorporated into this section by reference. In some embodiments, the acute wound is a full-thickness wound or a deep partial-thickness wound, and the wound type is a surgical wound, a burn wound, a bite wound, a puncture wound, a laceration or a wound from a sharp cut. In some embodiments, the acute wound is a deep partial-thickness burn wound, or a full-thickness burn. In some embodiments, the acute wound is a deep partial-thickness thermal burn wound, or a full-thickness burn wound. In some embodiments, the acute wound is a deep partial-thickness surgical wound, or a full-thickness surgical wound. Typically, in each of the above embodiments, the wound is a debrided wound where autografting is clinically indicated, and the depth of the wound is assessed at the time application.

In some embodiments, the wound is a chronic wound. Exemplary acute wounds are detailed in Section III, and incorporated into this section by reference.

Suitable subjects are detailed in Section IV, and incorporated into this section by reference. In some embodiments, the subject is 18-60 years of age, 18-65 years of age, 18-70 years of age, or 18-75 years of age. In some embodiments, the subject is less than ≥ 60 years of age, ≥ 65 years of age, ≥ 70 years of age, or ≥ 75 years of age. In some embodiments, the subject is less than 18 years of age, ≤ 15 years of age, or ≤ 10 years of age. In some embodiments, the subjects is ≤ 5 years of age, ≤ 3 years of age, ≤ 2 years of age, or ≤ 1 year of age. In some embodiments, the subject has limited surface area available for donor sites. In some embodiments, harvest of donor sites is contraindicated in the subject. In some embodiments, the subject is not hemodynamically stable. In some embodiments, the subject is at risk for delayed or impaired wound healing. In some embodiments, the subject has any combination of limited surface area available for donor sites, a contraindication for harvest of donor sites, hemodynamic instability, or a risk for delayed or impaired wound healing.

Methods of the present disclosure for wound closure may provide comparable or improved wound outcomes, as compared to treatment of comparable wound with autograft alone (“comparator autograft”), wherein the comparator autograft has a mesh ratio that is the same or less than the autograft overlaid with the skin substitute. A comparable outcome is an outcome that does not have a statistically significant difference. An improved out is an outcome that has a statistically significant difference (improvement). In various embodiments, a comparable or improved outcome may be comparable or decreased pain, comparable or improved cosmetic quality/qualities, comparable or decreased treatment associated adverse events, comparable or decreased scarring, comparable or decreased infection rate, comparable or decreased wound-infection related events, or any combination thereof, as assessed by an observer or by the subject, as compared to a comparable wound treated with autograft alone (“comparator autograft”), wherein the comparator autograft has a mesh ratio that is the same or less than the treatment autograft. In some embodiments, a method of the present disclosure provides comparable or improved vascularity, comparable or improved pigmentation, comparable or decreased thickness, comparable or decreased pain, comparable or increased pliability, comparable or increased scarring surface area, comparable or decreased stiffness, comparable or decreased itching, comparable or improved color, or any combination thereof, as assessed by an observer or by the subject, as compared to a comparable wound treated with autograft alone (“comparator autograft”), wherein the comparator autograft has a mesh ratio that is the same or less than the treatment autograft. Further details are provided in Section VI.

In an exemplary embodiment, the present disclosure provides a method for closing a wound of a subject, the method comprising applying to the wound an autograft with a mesh ratio of at least 2:1 and a skin substitute over the autograft, wherein the skin substitute is an organotypic human skin equivalent comprising NIKS cells, and wherein the wound is a deep partial-thickness burn wound or a full-thickness burn wound. In various embodiments, the subject may have limited surface area available for a donor site, be contraindicated for autografting, be hemodynamically unstable, be at risk for delayed or impaired wound healing, or any combination thereof. In still further embodiments, the burn may be a thermal burn. In still further embodiments, donor skin utilization is decreased by at least about 25%, 50%, 75%, 90%, 95%, or more. In certain exemplary embodiments of the above, the method comprises only a single application of the skin substitute.

In another exemplary embodiment, the present disclosure provides a method for closing a wound of a subject, the method comprising applying to the wound an autograft with a mesh ratio of at least 2:1and a skin substitute over the autograft, wherein the skin substitute is an organotypic human skin equivalent comprising NIKS cells, and wherein the wound is a deep-partial thickness burn wound or a full-thickness burn wound, and the % TBSA is ≥ 15%, ≥ 20%, or ≥ 25% and/or the surface of the wound is ≥ 200 cm², ≥ 300 cm², ≥ 400 cm², or ≥ 500 cm². In various embodiments, the subject may have limited surface area available for a donor site, be contraindicated for autografting, be hemodynamically unstable, be at risk for delayed or impaired wound healing, or any combination thereof. In still further embodiments, the burn may be a thermal burn. In still further embodiments, donor skin utilization is decreased by at least about 25%, 50%, 75%, 90%, or 95%, or more. In certain exemplary embodiments of the above, the method comprises only a single application of the skin substitute.

In another exemplary embodiment, the present disclosure provides a method for closing a wound of a subject, the method comprising applying to the wound an autograft with a mesh ratio of at least 2:1and a skin substitute over the autograft, wherein the skin substitute is an organotypic human skin equivalent comprising NIKS cells, and wherein the wound is a deep-partial thickness burn wound or a full-thickness burn wound, and the % TBSA is ≥ 25%, ≥ 35%, or ≥ 50% and/or the surface of the wound is ≥ 900 cm², ≥ 1000 cm², ≥ 2000 cm², ≥ 5000 cm², or ≥ 10,000 cm². In various embodiments, the subject may have limited surface area available for a donor site, be contraindicated for autografting, be hemodynamically unstable, be at risk for delayed or impaired wound healing, or any combination thereof. In still further embodiments, the burn may be a thermal burn. In still further embodiments, donor skin surface area is decreased by at least about 25%, 50%, 75%, 90%, or 95%. In certain exemplary embodiments of the above, the method comprises only a single application of the skin substitute.

In another exemplary embodiment, the present disclosure provides a method for wound closure, the method comprising applying to a wound of a subject less than 18 years of age an autograft with a mesh ratio of at least 2:1 and a skin substitute overlay, wherein the skin substitute is an organotypic human skin equivalent comprising NIKS cells, and wherein the wound is a deep-partial thickness burn wound or a full-thickness burn wound. In various embodiments, the subject may be about 15 years of age or less, about 10 years of age or less, about 5 years of age or less, or about 3 years of age or less. In still further embodiments, the burn may be a thermal burn. In still further embodiments, donor skin utilization is decreased by at least about 25%, 50%, 75%, 90%, 95%, or greater. In certain exemplary embodiments of the above, the method comprises only a single application of the skin substitute.

In another exemplary embodiment, the present disclosure provides a method for wound closure, the method comprising applying to a wound of a subject 65 years of age or greater an autograft with a mesh ratio of at least 2:1 and a skin substitute overlay, wherein the skin substitute is an organotypic human skin equivalent comprising NIKS cells, and wherein the wound is a deep-partial thickness burn wound or a full-thickness burn wound. In various embodiments, the subject may have limited surface area available for a donor site and/or be at risk for delayed or impaired wound healing. In still further embodiments, the burn may be a thermal burn. In still further embodiments, donor skin utilization is decreased by at least about 25%, 50%, 75%, 90%, 95%, or greater. In certain exemplary embodiments of the above, the method comprises only a single application of the skin substitute.

In another exemplary embodiment, the present disclosure provides a method for wound closure, the method comprising applying to a wound of a subject 75 years of age or greater an autograft with a mesh ratio of at least 2:1 and a skin substitute overlay, wherein the skin substitute is an organotypic human skin equivalent comprising NIKS cells, and wherein the wound is a deep-partial thickness burn wound or a full-thickness burn wound. In various embodiments, the subject may have limited surface area available for a donor site and/or be at risk for delayed or impaired wound healing. In still further embodiments, the burn may be a thermal burn. In still further embodiments, donor skin utilization is decreased by at least about 25%, 50%, 75%, 90%, 95%, or more. In certain exemplary embodiments of the above, the method comprises only a single application of the skin substitute.

In another exemplary embodiment, the present disclosure provides a method for wound closure, the method comprising applying to a wound of a subject at risk of impaired or delayed wound healing an autograft with a mesh ratio of at least 2:1 and a skin substitute overlay, wherein the skin substitute is an organotypic human skin equivalent comprising NIKS cells, and wherein the wound is a deep-partial thickness burn wound or a full-thickness burn wound. In various embodiments, the subject at risk for delayed or impaired wound healing may be a subject with a history of hypertrophic scarring, thinning dermis, inhalation injury or other coexisting injury, ≥ 25% TBSA, diabetes, infection (typically at the wound site but may be elsewhere), obesity, medications known to impair wound healing, a smoking habit, excessive alcohol drinking, low blood pressure, vascular disease, edema, cancer, malnutrition, or any combination thereof. In further embodiments, the subject may be < 18 years of age, ≤ 10 years of age, ≤ 5 years of age, ≤ 3 years of age, ≤ 2 years of age, ≤ 1 year of age, ≥ 60 years of age, ≥ 65 years of age, or ≥ 70 years of age. In still further embodiments, the burn may be a thermal burn. In still further embodiments, donor skin utilization is decreased by at least about 25%, 50%, 75%, 90%, 95% or more. In certain exemplary embodiments of the above, the method comprises only a single application of the skin substitute.

### VI. Improved Outcomes

In another aspect, the present disclosure provides a method for improving an outcome of wound healing, the method comprising applying to a wound of a subject a meshed autograft and a skin substitute overlay, wherein the skin substitute is an organotypic human skin equivalent comprising NIKS cells. In some embodiments, the autograft has a mesh ratio of at least 2:1. In some embodiments, the autograft has a mesh ratio of about 2:1 to about 8:1, about 3:1 to about 8:1, or about 4:1 to about 8:1. In some embodiments, the autograft has a mesh ratio of about 2:1 to about 6:1, about 3:1 to about 6:1, or about 4:1 to about 6:1. In each of the above embodiments, the skin substitute may be unmeshed or have mesh ratio of about 1:1 or less. The improved outcome may be measured by an observer (e.g., a medical provider) or by the subject, as compared to a comparable wound treated with autograft alone (“comparator autograft”), wherein the comparator autograft has a mesh ratio that is the same or less than the autograft overlaid with the skin substitute.

Wounds are typically prepared for treatment by methods known in the art, for instance cleansing with soap and water, surgical excision (fascial excision or tangential excision) if clinically indicated and feasible, etc. The location of the wound is not limiting. For instance, a wound may be on an arm, a leg, a torso, a face, a head, a neck, a hand, a foot, a buttock, over joints, etc. The meshed autograft and skin substitute may be applied to the wound within hours or days of the wound’s formation, within the first or second week of the wound’s formation, or even later. When the total surface area of the wound covered by the autograft is less than the manufactured size of a skin substitute, the skin substitute may be trimmed to substantially cover the surface area and shape of the wound area covered by the autograft. When the total surface area of the wound covered by the autograft is greater than the manufactured size of a skin substitute, multiple samples may be used to substantially cover the wound area covered by the autograft by abutting the multiple samples of the skin substitute. For the avoidance of doubt, the use of multiple samples to cover an autograft is considered a single application. In some embodiments, only a single application of the skin substitute is needed. In other embodiments, the method comprises multiple applications of the skin substitute to the wound area covered by the autograft or a portion of the wound area covered by the autograft. Depending upon the total surface area of the wound, allogenic tissue, autologous tissue without a skin substitute overlay, or autologous cells may be used in some embodiments in conjunction with the autograft and skin substitute overlay. For example, a wound area may be covered by abutting one or more samples of the autograft and skin substitutes overlay, and one or more autografts without a skin substitute overlay.

In various embodiments, an improved outcome may be a reduction in the incidence of autografting, decreased donor skin utilization, decreased pain, an improvement in one or more cosmetic quality of a closed wound, decreased treatment associated adverse events, decreased infection rate, decreased wound-infection related events, decreased donor-site morbidities, reduced health care resource utilization, improved quality of life, or combinations thereof. Qualities of a closed wound that may improve include, but are not limited to, pigmentation or coloring, scar thickness, pliability, stiffness, itching, vascularity, sensitivity to external stimuli (e.g., pain, touch, temperature, etc.) and overall appearance. Donor-site morbidities include but are not limited to tenderness, pain, cold-sensitivity, scarring in general or more particularly hypertrophic scarring, infection, conversion from a split-thickness wound to a complex wound or a full thickness wound, etc. In certain embodiments, an improved outcome may be a decrease in the incidence of autografting, decreased donor skin utilization, decreased pain, decreased scarring, improved pigmentation or coloring at a graft site, or any combination thereof. In certain embodiments, an improved outcome may be a decrease in the incidence of autografting, decreased donor skin utilization, and/or decreased pain. The improved outcome may be measured by an observer (e.g., a medical provider) or by the subject, as compared to a comparable wound treated with autograft alone (“comparator autograft”), wherein the comparator autograft has a mesh ratio that is the same or less than the autograft overlaid with the skin substitute.

A reduction in the incidence of autografting refers to a decrease in the number of times re-grafting needs to occurs due to failure of the initial autograft whether entirely or partially. A reduction in the incidence of autografting may be relative to a comparable wound treated with only an autograft of the same mesh ratio or of a similar mesh ratio (e.g., m-0.5 or m-1). In embodiments where an improved outcome includes a reduction in the incidence of autografting, there may be a 5%, 10%, 15%, 20%, 25%, 30%, 35%, 40%, 50%, 55%, 60%, 70%, 75%, 80%, 85%, 90%, 95%, or 100% decrease.

Decreased donor site utilization refers to a decrease in the total surface area of donor site(s) harvested from a subject, inclusive of donor sites harvested for an initial application and subsequent re-applications (if any). A decrease in donor site utilization may be relative to a comparable wound treated with only an autograft of the same mesh ratio. Alternatively, a decrease in donor site utilization may be relative to a comparable wound treated with only an autograft having a lower mesh ratio (e.g., m-1, m-2, m-3, etc.), but wherein both wounds have a similar outcome such as complete wound closure by 1, 2, 3 months, or durable would closure by 4, 5, 6 months, or one or more cosmetic quality of a closed wound (e.g., thickness, pliability, coloring, pain, itchiness, etc.). In embodiments where an improved outcome includes decreased donor site utilization, there may be about a 1.5-fold, about a 2-fold, about a 2.5-fold, about a 3-fold, about a 3.5-fold, about a 4, about a 4.5-fold, or about a 5-fold decrease.

Pain is a patient reported outcome, and may be evaluated by a variety of well-known, clinically tested pain assessment tools. Non-limiting examples include the visual analog scale and the Wong-Baker FACES pain rating scale.. A decrease in pain may be relative to a comparable wound treated with only an autograft having a lower mesh ratio (e.g., m-1, m-2, m-3, etc.), but wherein both wounds have a similar outcome such as complete wound closure by 1, 2, 3 months, or durable would closure by 4, 5, 6 months, or one or more cosmetic quality of a closed wound (e.g., scarring, pliability, coloring, pain, itchiness, etc.). One contributor to decreased pain associated with methods of the present disclosure is decreased autografting, which results in fewer donor sites. Decreased pain at the graft site may also contribute to an overall decrease in pain. Pain may also be assessed and reported locally (e.g., only at the graft site.) In embodiments where an improved outcome includes decreased pain, there may be a 1%, 5%, 10%, 15%, 20%, 25%, 30%, 35%, 40%, 50%, 55%, 60%, 70%, 75%, 80%, 85%, 90%, 95%, or 100% decrease in pain. In certain embodiments, there may be about a 5% to about a 50% decrease in pain, or about a 15% to about a 50% decrease in pain, about a 25% to about a 50% decrease in pain, or about a 35% to about a 50% decrease in pain. In other embodiments, there may be about a 25% to about a 75% decrease in pain, or about a 35% to about a 75% decrease in pain, about a 45% to about a 75% decrease in pain, or about a 55% to about a 75% decrease in pain. In still other embodiments, there may be about a 50% to about a 100% decrease in pain, about a 60% to about a 100% decrease in pain, about a 70% to about a 100% decrease in pain, or about a 80% to about a 100% decrease in pain. In further embodiments, there may be about a 90% to 100% decrease in pain, or even a 100% decrease in pain. In embodiments where an improved outcome includes an improvement in one or more cosmetic quality of a closed wound, the evaluation may take into consideration all wound sites, donor sites only, or graft sites only. An improvement in one or more cosmetic quality of a closed wound may be relative to a comparable wound treated with only an autograft of the same mesh ratio or a similar mesh ratio (e.g., m-0.5 or m-1). To evaluate a quality of a closed wound, preferably one or more parameter is compared to normal skin on a comparable anatomic location. Non-limiting parameters include overall opinion, vascularity, pigmentation or color, scar thickness, relief, pliability, scar surface area, pain associated with a scar, and itchiness. In describing these parameters below, various tests are described which an assessor may use. However, when the evaluator is a patient, the evaluation may be subjective. Vascularity refers to the presence of vessels in a closed wound or scar tissue assessed by the amount of redness. It may be tested by the amount of blood return after blanching with a piece of Plexiglas (or an equivalent thereof). Pigmentation refers to brownish coloration of tissue by pigment (melanin). It may be tested by applying Plexiglas (or an equivalent thereof) to the skin with moderate pressure to eliminate the effect of vascularity. For patient assessments, “scar color” is typically evaluated. Thickness refers to an average distance between the subcutical-dermal border and the epidermal surface of a scar. Relief refers to the extent to which surface irregularities are present (preferably compared with adjacent normal skin). Pliability refers to suppleness of a wound or scar tested by wrinkling an area between the thumb and index finger. Surface area refers to surface area of a scar in relation to the original wound area. Overall opinion refers to the overall opinion of the wound area or scar, which may or may not be based the parameters listed above. In an exemplary embodiment, the Patient and Observer Scar Assessment Scale (POSAS) or Patient Scar Assessment Questionnaire (PSAQ) is used evaluate scarring. Accordingly, an improvement in one or more cosmetic quality of a closed wound may be generally referred to as a decrease in scarring. In embodiments where the improved outcome includes an improvement in one or more cosmetic quality of a closed wound, there may be a 1%, 5%, 10%, 15%, 20%, 25%, 30%, 35%, 40%, 50%, 55%, 60%, 70%, 75%, 80%, 85%, 90%, 95%, or 100% decrease in scarring. In certain embodiments, there may be about a 5% to about a 50% decrease in scarring, or about a 15% to about a 50% decrease in scarring, about a 25% to about a 50% decrease in scarring, or about a 35% to about a 50% decrease in scarring. In other embodiments, there may be about a 25% to about a 75% decrease in scarring, or about a 35% to about a 75% decrease in scarring, about a 45% to about a 75% decrease in scarring, or about a 55% to about a 75% decrease in scarring. In still other embodiments, there may be about a 50% to about a 100% decrease in scarring, about a 60% to about a 100% decrease in scarring, about a 70% to about a 100% decrease in scarring, or about a 80% to about a 100% decrease in scarring. In further embodiments, there may be about a 90% to 100% decrease in scarring, or even a 100% decrease in scarring.

In embodiments where the improved outcome is decreased infection, there may be a 1%, 5%, 10%, 15%, 20%, 25%, 30%, 35%, 40%, 50%, 55%, 60%, 70%, 75%, 80%, 85%, 90%, 95%, or 100% decrease in infection. The evaluation may take into consideration infection at all treatment sites, at donor sites only, or at graft sites only. In certain embodiments, there may be about a 1% to about a 25% decrease in infection, about a 1% to about a 15% decrease in infection, about a 1% to about a 10% decrease in infection, or about a 1% to about a 5% decrease in infection. In other embodiments, there may be about a 5% to about a 50% decrease in infection, or about a 15% to about a 50% decrease in infection, about a 25% to about a 50% decrease in infection, or about a 35% to about a 50% decrease in infection. In other embodiments, there may be about a 25% to about a 75% decrease in infection, or about a 35% to about a 75% decrease in infection, about a 45% to about a 75% decrease in infection, or about a 55% to about a 75% decrease in infection. In still other embodiments, there may be about a 50% to about a 100% decrease in infection, about a 60% to about a 100% decrease in infection, about a 70% to about a 100% decrease in infection, or about a 80% to about a 100% decrease in infection. In further embodiments, there may be about a 90% to 100% decrease in infection, or even a 100% decrease in infection. One contributor to decreased infection associated with methods of the present invention is decreased donor skin utilization, which results in fewer donor sites (i.e., open wounds). Decreased infection at the graft site may also contribute to the overall decrease in infection rate.

Quality of life is a patient reported outcome, and may be evaluated by a variety of well-known, clinically tested burn specific or generic assessment tools. Non-limiting examples include the Sickness Impact Profile, the Burn Specific Health Scale-Brief (BSHS-B), the Medical Outcome Study Short Form-36 items (SH-36), the EuroQol five dimensions questionnaire (EQ-5D), Burn Specific Health Scale-Abbreviated (BSH-A), Burn Specific Health Scale-Revised 15D (BSHS-R), Quality of Life Questionnaire (QLQ), and the like. An increase in quality of life may be relative to a comparable wound treated with only an autograft of the same mesh ratio or of a similar mesh ratio (e.g., m-0.5 or m-1). In embodiments where an improved outcome includes increased quality of life (QOL), there may be a 1%, 5%, 10%, 15%, 20%, 25%, 30%, 35%, 40%, 50%, 55%, 60%, 70%, 75%, 80%, 85%, 90%, 95%, 100% or more increase in the QOL score. In certain embodiments, there may be about a 5% to about a 50% increase in QOL score, or about a 15% to about a 50% increase in QOL score, about a 25% to about a 50% increase in QOL score, or about a 35% to about a 50% increase in QOL score. In other embodiments, there may be about a 25% to about a 75% increase in QOL score, or about a 35% to about a 75% increase in QOL score, about a 45% to about a 75% increase in QOL score, or about a 55% to about a 75% increase in QOL score. In still other embodiments, there may be about a 50% to about a 100% increase in QOL score, about a 60% to about a 100% increase in QOL score, about a 70% to about a 100% increase in QOL score, or about a 80% to about a 100% increase in QOL score. In further embodiments, there may be about a 90% to 100% increase in QOL score, or even a 100% increase in QOL score.

Exemplary acute wounds are detailed in Section III, and incorporated into this section by reference. In some embodiments, the acute wound is a full-thickness wound, a partial-thickness wound, or a complex wound that contains intact dermal elements and the wound type is a surgical wound, a burn wound, a bite wound, a puncture wound, a laceration or a wound from a sharp cut. In some embodiments, the acute wound a partial-thickness burn wound, or a complex burn wound that contains intact dermal elements. In some embodiments, the acute wound a partial-thickness thermal burn wound, or a complex thermal burn wound that contains intact dermal elements. In some embodiments, the acute wound a partial-thickness surgical wound, or a complex surgical wound that contains intact dermal elements. Typically, in each of the above embodiments, the wound is a debrided wound where autografting is clinically indicated.

In some embodiments, the wound is a chronic wound. Exemplary acute wounds are detailed in Section III, and incorporated into this section by reference.

Suitable subjects are detailed in Section IV, and incorporated into this section by reference. In some embodiments, the subject is 18-60 years of age, 18-64 years of age, or 18-65 years of age. In some embodiments, the subject is less than ≥ 60 years of age, or ≥ 65 years of age. In some embodiments, the subject is less than 18 years of age, ≤ 15 years of age, or ≤ 10 years of age. In some embodiments, the subjects is ≤ 5 years of age, ≤ 3 years of age, ≤ 2 years of age, or ≤ 1 year of age. In some embodiments, the subject has limited surface area available for donor sites (e.g., young, elderly, ≥ 25% TBSA, etc.). In some embodiments, harvest of donor sites is contraindicated in the subject. In some embodiments, the subject is not hemodynamically stable. In some embodiments, the subject is at risk for delayed or impaired wound healing. In some embodiments, the subject has any combination of limited surface area available for donor sites, a contraindication for harvest of donor sites, hemodynamic instability, or a risk for delayed or impaired wound healing.

In one exemplary embodiment, the present disclosure provides a method for improving an outcome of wound healing, the method comprising applying to a wound of a subject an autograft with a mesh ratio of at least 2:1 and a skin substitute overlay, wherein the skin substitute is an organotypic human skin equivalent comprising NIKS cells, and wherein the wound is a deep-partial thickness burn wound or a full-thickness burn wound, and wherein the subject has a % TBSA of at least 10% and wherein the improved outcome is a decrease in donor skin utilization of at least 30%, at least 40%, or at least 50%, as compared to a comparable wound treated with autograft alone (“comparator autograft”), wherein the comparator autograft has a mesh ratio that is the same as the autograft overlaid with the skin substitute.

In another exemplary embodiment, the present disclosure provides a method for improving an outcome of wound healing, the method comprising applying to a wound of a subject an autograft with a mesh ratio of at least 2:1 and a skin substitute overlay, wherein the skin substitute is an organotypic human skin equivalent comprising NIKS cells, and wherein the wound is a deep-partial thickness burn wound or a full-thickness burn wound, and wherein the subject has a % TBSA of at least 20% and wherein the improved outcome is a decrease in donor skin utilization of at least 30%, at least 40%, or at least 50%.

In another exemplary embodiment, the present disclosure provides a method for improving an outcome of wound healing, the method comprising applying to a wound of a subject an autograft with a mesh ratio of at least 2:1 and a skin substitute overlay, wherein the skin substitute is an organotypic human skin equivalent comprising NIKS cells, and wherein the wound is a deep-partial thickness burn wound or a full-thickness burn wound, and wherein the subject has a % TBSA of at least 30% and wherein the improved outcome is a decrease in donor skin utilization of at least 30%, at least 40%, or at least 50%.

In another exemplary embodiment, the present disclosure provides a method for improving an outcome of wound healing, the method comprising applying to a wound of a subject an autograft with a mesh ratio of at least 2:1 and a skin substitute overlay, wherein the skin substitute is an organotypic human skin equivalent comprising NIKS cells, and wherein the wound is a deep-partial thickness burn wound or a full-thickness burn wound, and wherein the subject has a % TBSA of at least 40% and wherein the improved outcome is a decrease in donor skin utilization of at least 30%, at least 40%, or at least 50%.

In another exemplary embodiment, the present disclosure provides a method for improving an outcome of wound healing, the method comprising applying to a wound of a subject an autograft with a mesh ratio of at least 2:1 and a skin substitute overlay, wherein the skin substitute is an organotypic human skin equivalent comprising NIKS cells, and wherein the wound is a deep-partial thickness burn wound or a full-thickness burn wound, and wherein the subject has a % TBSA of at least 50% and wherein the improved outcome is a decrease in donor skin utilization of at least 20%, at least 30%, or at least 40%.

So that the present invention may be more readily understood, certain terms are defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ± 5%, but can also be ± 4%, 3%, 2%,1%, etc. Whether or not modified by the term “about,” the claims include equivalents to the quantities.

### EXAMPLES

The following examples illustrate various iterations of the invention.

### Example 1

A Phase ½a, multicenter, open-label, randomized, within-patient, controlled study is evaluating the efficacy, safety, and tolerability of StrataGraft® overlay of meshed autograft (SOMA) in treatment of full-thickness thermal burns. Study participants will receive meshed autograft on one part of their burn (AG Tx) and meshed autograft with a StrataGraft® covering (SOMA Tx) on a different part of their burn. When collectively referring to both study treatments (i.e., the SOMA Tx and the AG Tx), the term “Study Tx” is used.

Eligible subjects will be aged 18-75 years with thermal burns of 10%-49% (inclusive) total body surface area (TBSA), including areas of full thickness injury clinically indicated for surgical excision and autografting. On each subject, 2 full thickness wounds, comparable in area, wound bed composition (fat, fascia, or muscle), and kinesiologic stressors are designated as Sites A and B. Sites A and B will be randomized to receive either AG Tx or SOMA Tx, such that each subject receives both treatments. In this way, subjects serve as their own controls. Sites A and B are 100-1900 cm², on torso or limbs, and may cross joints.

Following excision, the AG Tx site receives meshed split-thickness autograft (mesh ratio “m”, per clinical judgment) while the SOMA Tx site receives split-thickness autograft that is meshed at 1 or 2 mesh ratio(s) greater than the AG Tx site (m+1 or m+2) and overlaid with StrataGraft® skin tissue meshed 1:1. Autografting will occur on day 1. Autograft will be secured in place using staples, sutures, or adhesive, based on the investigator’s discretion. StrataGraft® will be trimmed to the approximate size of the autograft and secured in place using staples, sutures, or adhesive, based on the investigator’s discretion. Blinded assessment of all treated wounds will begin on day 7±1 day, continue daily until the wound is confirmed closed, and then at each visit for the duration of that subject’s study participation (e.g., day 14±1 day, day 21±2 day, day 28±3 day, day 42±4 day, month 2±4 day, month 3±7 day, month 6±2 weeks, month 12±4 weeks).

StrataGraft® is manufactured by Stratatech (Madison, WI) in compliance with Good Manufacturing Practice (GMP) via proprietary processes. StrataGraft® is cryopreserved, shipped deep frozen on dry ice to each study site, and stored at -70° to -90° C. prior to use.

Efficacy assessments associated with wound healing, for the study, include donor skin utilization, re-epithelialization, complete wound closure, durable wound closure, Patient and Observer Scar Assessment Scale (POSAS) assessments, and Patient Scar Assessment Questionnaire (PSAQ) assessments. Complete wound closure for the study is defined as complete skin re-epithelialization without drainage confirmed at 2 visits at least 2 weeks apart. Durable wound closure for the study is defined as persistence of closure, maintained for at least 3 months after the initial observation of closure (FDA. Guidance for Industry: Chronic cutaneous ulcer and burn wounds – developing products for treatment. June 2006). Cosmesis of treatment and donor sites is assessed by both clinical observers and subjects using POSAS and/or PSAQ. Subjects and observers separately assess 6 cosmetic categories and an overall opinion, each ranked on a scale from 1 (normal skin) to 10 (worst imaginable), and then category scores are summed to create a total score from each assessor. Observer assessments include categories of vascularity, pigmentation, thickness, relief, pliability, and surface area, as well as an overall opinion score. Patient assessments include categories of pain, itching, color, stiffness, thickness, and irregularity, as well as an overall opinion score.

One endpoint is difference in percent of SOMA Tx sites and AG Tx sites with complete closure at day 28±3 day. Another endpoint is difference in total score of observer assessment (POSAS) of SOMA Tx sites and AG Tx sites at day 28±3 day. Another endpoint is calculated percent reduction of donor skin (as defined by [1-(AG Tx mesh ratio/SOMA Tx mesh ratio) X 100]) at Month 2. Another endpoint is difference in percent of SOMA Tx sites and AG Tx sites with complete wound closure without further autografting at Month 2. Additional endpoints may include incidence and/or percent re-epithelializatioin of the Study Tx sites at day 14±1 day, day 21±2 day, day 28±3 day, day 42±4 day, month 2±4 day, month 3±7 day; percent subjects with durable wound closure of Study Tx sites at month 3±7 day, month 6±2 weeks, month 12±4 weeks; POSAS/PSAQ scores of Study Tx sites at day 28±3 day, month 2±4 day, month 3±7 day, month 6±2 weeks, and month 12±4 weeks; total area of donor skin applied to Study Tx sites at month 2±4 day, month 3±7 day, month 6±2 weeks, month 12±4 weeks. Another endpoint is the effect of total wound burden on rate of wound closure. It is expected the SOMA Tx site will perform comparable or superior to AG Tx site for one or more endpoint.

Other efficacy assessments for the study relate to health care resource utilization, including location of care for StrataGraft® applications (e.g., inpatient hospital, outpatient/ambulatory hospital, clinic, etc.), treatments used for all other non-study burn areas following excision and % total body surface area (TBSA) treated with each, number and duration of operating room procedures required for study burn treatment, length of hospital stay, prescription drugs given for pain control, antibiotics given for outpatient use at discharge, re-admission within 30 days after discharge, whether a re-admission is planned, and length of hospital stay after re-admission.

Safety assessments include monitoring of treatment-emergent adverse events (TEAEs), vital signs, laboratory parameters, incidence of wound infection and wound-infection related events at the Study Tx sites, donor-site complications, and immunologic responses to StrataGraft®. Each patient is assessed for an immune response to StrataGraft® via panel reactive antigen (PRA) at baseline and Month 3±7 day. PRA evaluation may be performed using solid-phase Luminex assays for CTHLA class I antibody and CL1 RFX HLA class II antibody. Patients with any PRA value >0% are considered to have a positive PRA response. Changes in immune status are monitored by comparing them with baseline values for each patient. At Month 3±7 day, patients have a 1 × 0.5 cm biopsy punch taken from a healed edge of the Study Tx site across the wound/normal skin border. The biopsy tissue will be examined using standard histologic techniques and assessed for tissue characteristics such as architecture, presence of immune cells, elastin and collagen distribution.

Safety endpoints may include incidence of treatment-emergent AEs (TEAEs) throughout study, incidence of TEAEs related to each of the Study Txs, incidence of wound infection-related events at Study Tx site, clinically significant changes in vital signs throughout study compared to baseline, clinically significant changes in laboratory values between Baseline and Day 28, clinically significant changes in panel reactive antibodies (PRA) and anti-bovine serum albumin (BSA) levels between baseline and Month 3, and clinically significant changes in concomitant medication use.

What is claimed is:

1. A method for treating a full-thickness wound in a subject, the method comprising applying to the wound an autograft with a mesh ratio of about 2:1 to about 8:1 (“treatment autograft”) and a skin substitute overlay, wherein the skin substitute is an organotypic human skin equivalent comprising NIKS cells.

2. A method for obtaining closure of a full-thickness wound in a subject, the method comprising applying to the wound an autograft with a mesh ratio of about 2:1 to about 8:1 (“treatment autograft”) and a skin substitute overlay, wherein the skin substitute is an organotypic human skin equivalent comprising NIKS cell and wherein wound closure is about 90% re-epithelialization or greater.

3. The method of claim 2, wherein wound closure occurs within about 12 weeks of initial application of the skin substitute.

4-6. (canceled)

7. The method of claim 2, wherein wound closure is about 95% re-epithelialization or greater.

8-9. (canceled)

10. The method of claim 7, wherein wound closure is complete wound closure.

11. The method of claim 7, wherein wound closure is durable wound closure.

12. The method of claim 2, wherein the closed wound has comparable or improved vascularity, comparable or improved pigmentation, comparable or decreased thickness, comparable or decreased pain, comparable or increased pliability, comparable or increased scarring surface area, comparable or decreased stiffness, comparable or decreased itching, comparable or improved color, or any combination thereof, as assessed by an observer or by the subject, as compared to a comparable wound treated with autograft alone (“comparator autograft”), wherein the comparator autograft has a mesh ratio that is the same or less than the treatment autograft.

13. A method for improving an outcome of wound healing in a subject, the method comprising: applying to a full-thickness wound of a subject an autograft with a mesh ratio of about 2:1 to about 8:1 (“treatment autograft”) and a skin substitute overlay, wherein the skin substitute is an organotypic human skin equivalent comprising NIKS cells;
wherein the improved outcome is a reduction in the incidence of autografting, decreased donor skin utilization, decreased pain, an improvement in one or more cosmetic quality of the closed wound, decreased treatment associated adverse events, decreased infection rate, decreased wound-infection related events, decreased donor-site morbidities, reduced health care resource utilization, improved quality of life, or combinations thereof, as measured by an observer or by the subject in comparison to a comparable wound treated with autograft alone (“comparator autograft”), wherein the comparator autograft has a mesh ratio that is the same or less than the treatment autograft.

14. The method of claim 13, wherein the improvement is statistically significant.

15. The method of claim 13, wherein the comparator autograft has a mesh ratio less than the treatment autograft.

16. The method of claim 1, wherein the treatment autograft has a mesh ratio of about 3:1 to about 8:1.

17-18. (canceled)

19. The method of claim 1, wherein the treatment autograft has a mesh ratio of about 2:1 to about 6:1.

20. (canceled)

21. The method of claim 1, wherein there is only a single application of the skin substitute to the wound.

22. The method of claim 1, wherein there is only a single application of the treatment autograft to the wound.

23. The method of claim 1, wherein the wound is a burn wound.

24. The method of claim 23, wherein the wound is an electrical burn wound, a chemical burn wound, or a thermal burn wound.

25. The method of claim 1, wherein the subject has a total wound area covering up to about 85% total body surface area (TBSA), the total wound area comprising the area of the wound.

26. The method of claim 25, wherein the subject has a full thickness % TBSA of about 10% to about 60%.

27. (canceled)

28. The method of claim 25, wherein the method further comprises applying the treatment autograft and the skin substitute overlay to a plurality of wounds.

29. The method of claim 1, wherein the skin substitute comprises a dermal equivalent, the dermal equivalent comprising gelled collagen containing normal human dermal fibroblasts.

30. The method of claim 29, wherein the collagen present in the dermal equivalent comprises type I murine collagen.

31. The method of claim 29, wherein the only collagen in the dermal equivalent is produced by cells of the skin substitute.

32. The method of claim 1, wherein the wound is clinically indicated for excision and grafting.

33-39. (canceled)

40. The method of claim 1, wherein the treatment autograft and the skin substitute are applied to a surface area of less than 200 cm².

41. The method of claim 1, wherein the treatment autograft and the skin substitute are applied to a surface area about 100 cm² or greater.

42-44. (canceled)

45. The method of claim 41, wherein the treatment autograft and the skin substitute are applied to a surface area of about 100 cm² to about 10,000 cm².

46. (canceled)

47. The method of claim 1, wherein the organotypic human skin equivalent comprising NIKS has a mesh ratio of about 1:1.
